(19) United States
(12) Patent Application Publication
(10) Pub. No.: US 2007/0265709 A1
(43) Pub. Date: Nov. 15, 2007

(54) Gastric Bypass Devices and Methods

(75) Inventors: Elizabeth Rajan, Rochester, MN (US); Christopher J. Gostout, Rochester, MN (US); Kevin E. Bennet, Rochester, MN (US)

Correspondence Address: MERCHANT & GOULD PC, P.O. BOX 2903, MINNEAPOLIS, MN, 55402-0903, US

(73) Assignee: Mayo Foundatio for Medical Education and Research, Rochester, MN (US)

(21) Appl. No.: 10/590,229

(22) PCT Filed: Feb. 24, 2005
(86) PCT No.: PCT/US05/05782

§ 371 Date: Jun. 28, 2007

Related U.S. Application Data
(60) Provisional application No. 60/547,483, filed on Feb. 25, 2004.

(51) Int. Cl. A61F 2/04

(52) U.S. Cl. 623/23.64

(57) ABSTRACT

Devices and methods for achieving weight loss in obese and morbidly obese patients are disclosed. The invention combines a gastric and/or small bowel bypass (in which ingested food bypasses the stomach and, optionally, at least a portion of the small bowel) and a satiety device in the form of one or more expandable chambers, that tend to induce a feeling of satiety after the patient has consumed a relatively small amount of food. This combination of gastric and/or small bowel bypass and satiety structure may be delivered and removed using minimally invasive procedures such as an endoscope. In some instances, the gastric and/or small bowel bypass device itself can form the satiety structure. In other devices, the gastric bypass may be independent of the satiety structure (which may be provided as one or more expandable chambers).

The present invention relates to the field of devices and methods for weight control.

A variety of medical approaches may be used to achieve weight loss or control in humans and/or animals. The approaches may include exercise, diets, medication, surgical procedures, etc. and combinations of two or more different approaches. Surgical procedures may include, e.g., removing part of the stomach and rearranging the small bowel, stapling part of the stomach and rearranging the small bowel, bypassing the stomach by rearranging the jejunum and making a pouch of the upper intestines.

Such procedures are, however, irreversible and may cause the patient to experience symptoms such as, e.g., indigestion, poor vitamin uptake, diarrhea, malnutrition, etc. In addition, these procedures necessarily involve some inherent risk to the patient as surgical procedures. In spite of these disadvantages, patients still proceed with the surgical approaches when faced with the need to achieve weight loss to address obesity.

### SUMMARY OF THE INVENTION

The present invention provides devices and methods for achieving weight loss in obese and morbidly obese patients. The invention combines a gastric and/or small bowel bypass (in which ingested food bypasses the stomach and, optionally, at least a portion of the small bowel) and a satiety device in the form of one or more expandable chambers, that tend to induce a feeling of satiety after the patient has consumed a relatively small amount of food.

This combination of gastric and/or small bowel bypass and satiety structure can provide significant advantages in achieving weight loss using a device that can preferably be delivered and removed using minimally invasive procedures such as an endoscope. In some instances, the gastric and/or small bowel bypass device itself can form the satiety structure. In other devices, the gastric bypass may be independent of the satiety structure (which may be provided as one or more expandable chambers).

In at least some embodiments, it may be preferred that the gastric and/or small bowel bypass and/or expandable chambers of the devices be somewhat permeable to gastric fluids or allow for their removal from the stomach. If gastric fluids are permitted to enter the gastric bypass, motility of ingested food therethrough may be enhanced. Other potential advantages of providing for the removal or drainage of at least some gastric fluids may include, e.g., reducing the likelihood of bezoars, etc.

It may be preferred that one or more components in the devices of the present invention be attached within the subject's gastrointestinal system to, e.g., maintain patency, prevent migration, etc. On the esophageal side, it may be preferred that the device be attached securely to the esophagus to guide ingested food into the gastric bypass. The gastric bypass and/or satiety device may be attached within the subject's stomach and/or a small bowel extension (if present) may be attached to the subject. Depending on the device, only one of the components (i.e., esophageal extension, gastric bypass, satiety device, and small bowel extension) may be attached or any combination of two or more of the components may be attached to the subject. Examples of attachments may include one or more techniques, e.g., endoscopic suturing, stapling, creating an outward flaring of the esophageal tube similar to that found in, e.g., enteral stents, etc.

On the small bowel side, it may be preferred that the devices of the present invention prevent or reduce contact between ingested food and the small bowel such that the absorption of nutrients from the ingested food can be reduced. In some instances, the devices may include a small bowel extension that extends from the pyloric sphincter to the duodenum or anywhere within the small bowel.

In one aspect, the present invention provides an implantable gastro-intestinal device including a gastric bypass having an inlet and an outlet; an expandable chamber attached to the gastric bypass; an optional esophageal extension attached to the inlet of the gastric bypass, wherein food passes into the gastric bypass through the esophageal extension; and an optional small bowel extension attached to the outlet of the gastric bypass, wherein the small bowel extension receives material exiting the outlet of the gastric bypass.

In another aspect, the present invention provides an implantable gastrointestinal device including a gastric bypass having an inlet and an outlet; an expandable chamber attached to an outer surface of the gastric bypass, wherein the expandable chamber comprises a plurality of adjacent subchambers in fluid communication with each other, wherein the plurality of adjacent subchambers are distributed over at least a portion of the outer surface of the gastric bypass, and wherein the plurality of adjacent subchambers maintain an open volume in the gastric bypass in the absence of compressive forces thereon; an optional esophageal extension attached to the inlet of the gastric bypass, wherein food passes into the gastric bypass through the esophageal extension; and an optional small bowel extension attached to the outlet of the gastric bypass, wherein the small bowel extension receives material exiting the outlet of the gastric bypass.

In another aspect, the present invention provides an implantable gastro-intestinal device including a gastric bypass having an inlet and an outlet; an expandable toroidal chamber located proximate the inlet of the gastric bypass, wherein the toroidal chamber holds the inlet in an open configuration when the toroidal chamber is inflated; an optional esophageal extension attached to the inlet of the gastric bypass, wherein food passes into the gastric bypass through the esophageal extension; and an optional small bowel extension attached to the outlet of the gastric bypass, wherein the small bowel extension receives material exiting the outlet of the gastric bypass.

These and other features and advantages of the present invention may be described below in connection with various exemplary embodiments of the devices and methods of the present invention.

### BRIEF DESCRIPTIONS OF THE FIGURES

FIG. 1A depicts one exemplary embodiment of one device according to the present invention in location within the gastrointestinal system of a patient.

FIG. 1B is a cross-sectional view of the device of FIG. 1A, taken along a plane parallel to the paper on which FIG. 1A appears.

FIG. 2 is a cross-sectional view of another exemplary embodiment of a device according to the present invention.

FIG. 3 is a partial cross-sectional view of a portion of another exemplary embodiment of a device according to the present invention.

FIG. 4 depicts an alternative exemplary embodiment of a device according to the present invention.

FIG. 5 is an enlarged cross-sectional view of a portion of the device of FIG. 4.

FIG. 6 depicts another alternative embodiment of the present invention.

### DETAILED DESCRIPTION OF EXEMPLARY EMBODIMENTS OF THE PRESENT INVENTION

In the following detailed description of some exemplary embodiments of the invention, reference is made to the accompanying figures of the drawing which form a part hereof, and in which are shown, by way of illustration, specific embodiments in which the invention may be practiced. It is to be understood that other embodiments may be utilized and structural changes may be made without departing from the scope of the present invention.

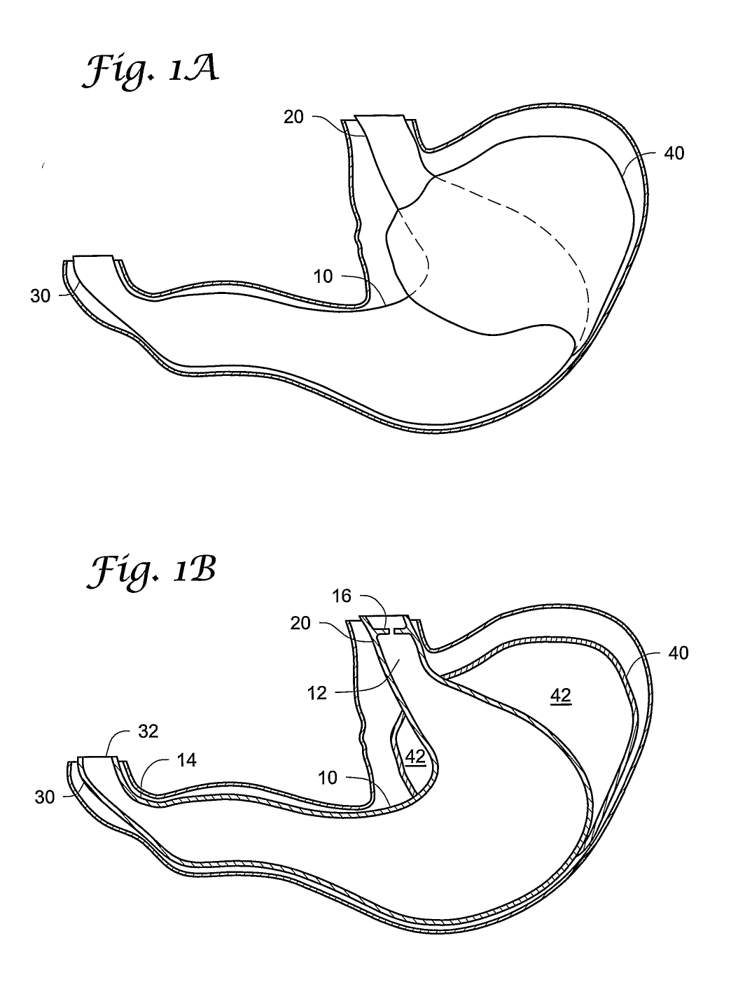

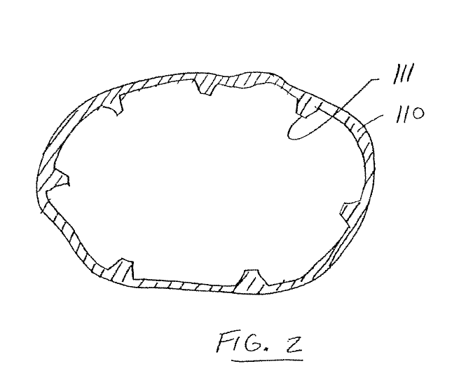

FIGS. 1A-1B depict a device according to the present invention in position in the gastrointestinal system of a patient. The device includes a gastric bypass 10 that includes an inlet 12 located proximate the esophagus and an outlet 14 located proximate the pyloric valve at the exit of the stomach and entrance to the duodenum. The device may preferably include one or more flaps 16 or other structures to prevent or reduce the likelihood of food re-entering the esophagus after passing into the gastric bypass 10.

The gastric bypass 10 may preferably be provided in the form of a gastric bag that is flexible such that normal movement of the stomach can compress and distort the gastric bag to improve the motility of ingested fluid therethrough. In some instances, it may be preferred that the gastric bypass 10 be in the form of a flaccid gastric bag, i.e., a flexible bag that has no self-supporting shape. It may also be preferred that the gastric bypass 10 exhibit some permeability to gastric fluids which may, e.g., improve the motility of ingested food, reduce the likelihood of bezoar formation in the stomach, etc.

The device may also preferably include an optional esophageal extension 20 attached to the inlet 12 of the gastric bypass 10. Food ingested by a patient preferably passes into the gastric bypass 10 through the esophageal extension 20.

In addition to directing ingested food into the gastric bypass 10, the esophageal extension 20 may also serve to anchor or fix the gastric bypass 10 in position relative to the esophagus. The esophageal extension 20 may also provide a location at which the device can be attached or fixed to the patient using, e.g., sutures, staples, etc.

An optional small bowel extension 30 is depicted in connection with the device, with the small bowel extension 30 attached to the outlet 14 of the gastric bypass 10. The optional small bowel extension 30 preferably receives material exiting the outlet 14 of the gastric bypass 10 and may also preferably prevent or at least reduce contact between that food and at least a portion of the small bowel. By reducing contact between ingested food and the small bowel, the amount of nutrition that can be obtained from the ingested food can be reduced. The small bowel extension 30 may preferably terminate at an end 32 located in the jejunem. It may also be preferred that the small bowel extension exhibit some permeability to duodenal juices that may, e.g., improve the motility of ingested food, reduce the likelihood of obstruction in the small bowel extension, etc.

The device depicted in FIGS. 1A-1B may also preferably include an expandable chamber 40 in the form of a toroid. The toroidal chamber 40 includes a volume 42 when inflated. The toroidal chamber 40 may serve one or more different functions in connection with the device. For example, the expanded chamber 40 may preferably hold the inlet 12 of the gastric bypass 10 open when expanded. By so doing, the chamber 40 may assist ingested food to pass from the esophageal extension 20 into the gastric bypass 10 through inlet 12 without significant restriction.

In addition to holding the inlet 12 of the gastric bypass 10 open, the chamber 40 may also serve to hold the remainder of the gastric bypass 10 open when the chamber 40 is expanded. In some embodiments, the expanded chamber 40 may serve to hold the gastric bypass 10 open to the pyloric region. The expanded chamber 40 may also limit or prevent twisting of the gastric bypass 10. Such limiting or prevention of twisting may be enhanced if the chamber 40 is in the shape of an asymmetric toroid, e.g., the expanded chamber 40 is larger on one side. One potential advantage of such a construction is that twisting or rotation of the chamber 40 itself within the stomach may be limited or prevented.

Another potential function of chamber 40 is to provide a volume-occupying structure that may tend to induce a feeling of satiation after the patient has consumed a relatively small amount of food.

The shape of the toroidal chamber 40 may vary from that of a regular toroid, i.e., the exact shaped of the toroidal chamber 40 may be adapted to correspond better to the shape of the stomach. For example, the toroidal chamber 40 may be enlarged on one side to better conform to the shape of the fundus.

FIG. 2 is a cross-sectional view of a portion of a gastric bypass 110 that may be provided in connection with the present invention. The construction of the depicted gastric bypass 110 may, e.g., be used in connection with the gastric bypass depicted and described above in connection with FIG. 1. The gastric bypass 110 may preferably include internal ribs 111 that may serve a number of purposes. For example, the ribs 111 may assist the gastric bypass 110 in maintaining an open internal in the absence of compressive forces thereon. In some embodiments, the ribs 111 may also assist the gastric bypass 110 to resist compression by, e.g., contractions of the stomach. The ribs 111 may extend lengthwise along the gastric bypass 110 (i.e., in direction generally aligned from the esophagus to the pyloric valve). In other embodiments, the ribs 111 may extend helically about the bag 110 or be provided in other configurations.

Also, although the ribs 111 are depicted as internal to the gastric bypass 110, it should be understood that in some embodiments, the ribs 111 may be located on the external surface of the bag. In other embodiments, ribs may be provided on both the internal surfaces and the external surfaces of the gastric bypass. In still other embodiments, ribs or stiffening structures may be encapsulated within the walls of the of the gastric bypass such that both the interior and the exterior surfaces of the gastric bypass are relatively smooth or flat surfaces.

The ribs 111 are depicted as being integral with the structure of the gastric bypass 110. It should, however, be understood that the ribs 111 may alternatively be attached to the bag 110 and may be made of the same or different materials as the gastric bag 110. In some instances, even ribs 111 that are integral with the gastric bypass 110 may be manufactured of materials that are different than the materials used to manufacture the gastric bypass 110 (e.g., through a coextrusion or other molding process).

Furthermore, similar ribs may be used in the optional esophageal extensions and/or small bowel extensions that may be provided in connection with the gastric bypass of the present invention. The ribs may provide structural support to assist in maintaining patency of the extensions, i.e., prevent complete closure, twisting, etc.

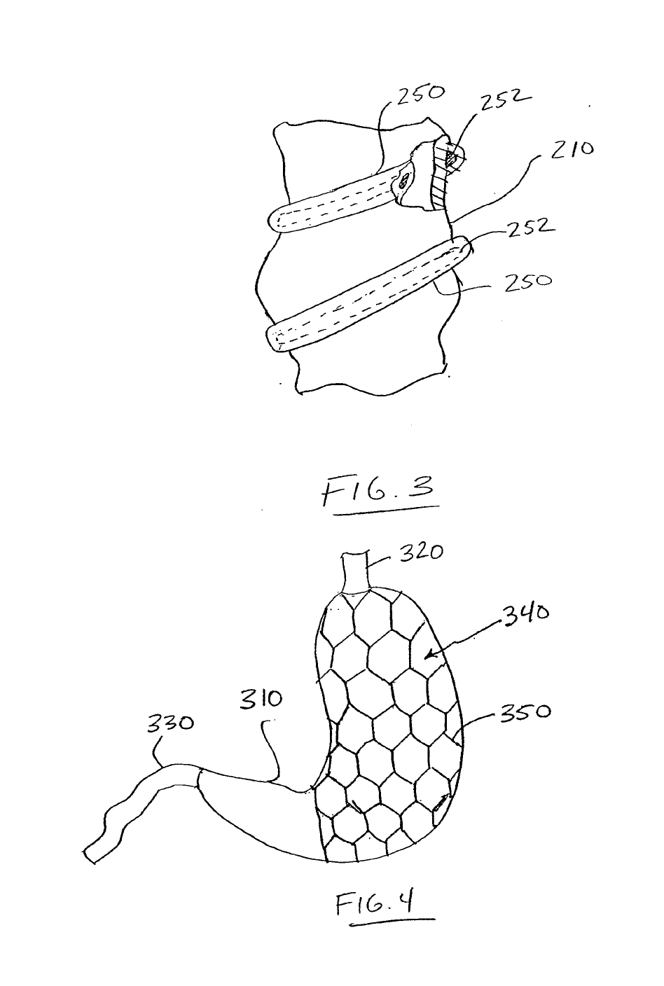

FIG. 3 depicts a portion of another gastric bypass 210 with a portion of the gastric bypass 210 cut-away to provide a partial cross-sectional view. The depicted gastric bypass 210 includes one or more sleeves 250, with the sleeves 250 including wires 252 located therein. The sleeves 250 and wires 252 may be provided to assist the gastric bypass 210 in maintaining an open internal volume in the absence of compressive forces. In addition, the sleeves 250 and wires 252 may assist the gastric bypass 210 in resisting compressive forces.

One potential use of sleeves 250 and wires 252 is that the gastric bypass 210 may be delivered to its deployment site with the gastrointestinal tract without the wires 252 in place within sleeves 250. One potential benefit of such a deployment method is that the gastric bypass 210 may have a lower profile and/or be more flexible to facilitate its deployment. With the gastric bypass 210 in position, the wires 252 may preferably be inserted endoscopically to expand the gastric bypass 210.

Although the sleeves are depicted on the exterior of the gastric bypass 210, it should be understood that they could, alternatively, be located in the interior of the gastric bypass 210. Also the gastric bypass 210 could include one sleeve 250 (and corresponding wire 252) that extends helically about the gastric bypass 210 or a plurality of sleeves 250 and wires 252 could be used. In another variation, one sleeve 250 could be provided with two or more wires inserted therein. In still another variation, a single wire could be inserted into a plurality of sleeves 250 provided on the gastric bypass 210.

Still another variation is that although the sleeves 250 and wires 252 are depicted in a generally helical arrangement relative to the gastric bypass 210, they could be provided in any suitable orientation, e.g., longitudinal (e.g., extending generally from the inlet to the outlet of the gastric bypass 210), annular (e.g., extending about the perimeter of the gastric bypass—in which case it might be preferred to provide a series of the annular sleeves and wires), or any other suitable arrangement (including combinations of different orientations (e.g., annular and longitudinal, etc.).

Furthermore, similar sleeves and wires may be used in the optional esophageal extensions and/or small bowel extensions that may be provided in connection with the gastric bypass of the present invention. The sleeves and wires may provide similar structural support to assist in maintaining patency of the extensions, i.e., prevent complete closure, twisting, etc.

FIG. 4 depicts another embodiment of a device according to the present invention in which a gastric bypass 310 preferably includes an esophageal extension 320 and a small bowel extension 330 as described in connection with, e.g., the embodiment of FIG. 1. The gastric bypass 310 also includes an expandable chamber 340 attached to an outer surface of the gastric bypass 310, wherein the expandable chamber 340 includes a plurality of adjacent subchambers 350 that may preferably be in fluid communication with each other. The adjacent subchambers 350 may preferably be distributed over at least a portion of the outer surface of the gastric bypass 310 such that the adjacent subchambers 350 maintain an open volume in the gastric bypass 310 in the absence of compressive forces thereon.

The subchambers 350 may be provided in a geodesic-like pattern as depicted in FIG. 4. Alternatively, the subchambers may take any other suitable shapes, e.g., a series of interconnected annular rings, helical spirals, circles, etc. In some instances, the subchambers may, within the area they are located, cover the entire surface of the gastric bypass 310. In other arrangements, spaces may be provided between the subchambers. The subchambers may also be provided in combinations of two or more different shapes.

It may be advantageous, in some embodiments, to provide one or more expandable chambers and/or subchambers on the optional esophageal and/or small bowel extensions to assist them in maintaining patency, i.e., prevent complete closure, twisting, etc.

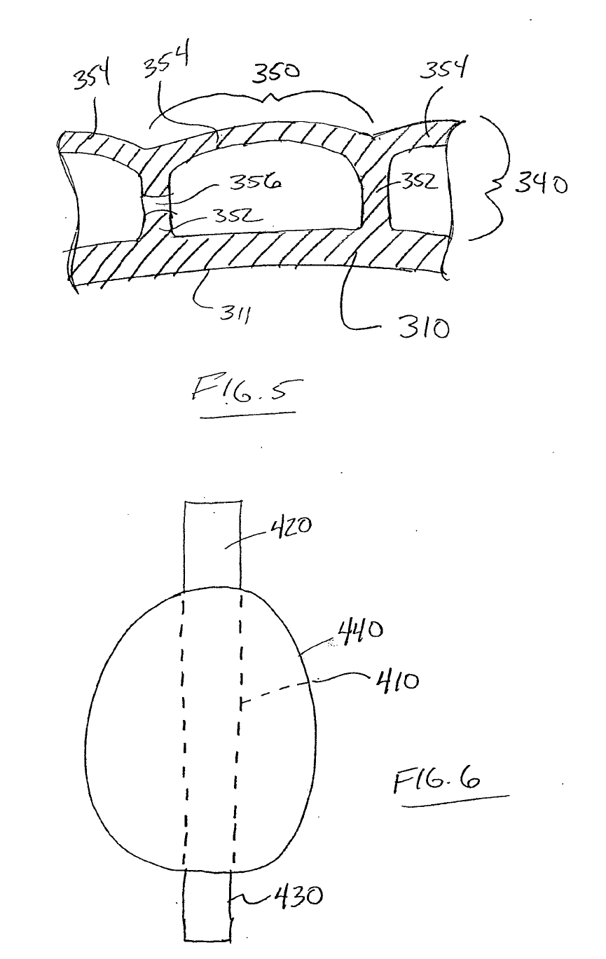

FIG. 5 is an enlarged cross-sectional view of a portion of the gastric bypass 310 and the associated expandable chamber 340 with its subchambers 350 depicting only one potential construction of the device. The expandable subchambers 350 may be defined by a series of webs 352 extending from the gastric bypass 310, with the webs 352 connected by membrane 354 to form the subchambers 350. The subchambers 350 are preferably in fluid communication with each other. One construction to achieve that fluid communication may include, e.g., openings 356 in one or more webs 352. Some alternative constructions may include webs 352 that are manufactured of permeable materials, etc.

Also, although the expandable chamber 340 is depicted as being located on the external surface of the gastric bypass 310 (with surface 311 being the internal surface of the gastric bypass 310), the expandable chamber 340 and its associated subchambers 350 may alternatively be located on the interior of the gastric bag 310 if so desired. In another variation, it should be understood that more than one expandable chamber 340 may be provided, with each expandable chamber 340 including two or more subchambers 350.

FIG. 6 depicts another exemplary embodiment of a device according to the present invention. The device includes a gastric bypass 410 that preferably includes an optional esophageal extension 420 and an optional small bowel extension 430 as described in connection with, e.g., the embodiment of FIG. 1. The device further includes a chamber 440 that may preferably be expandable to assist in retaining the device in place within the gastrointestinal system as well as to transmit compressive forces from the stomach to the gastric bypass 410 passing therethrough.

A variety of different exemplary embodiments of devices are described above. It should be understood that devices according to the present invention may include one or more of the different features described herein. For example, the gastric bypass 10 of FIG. 1 may include ribs 111 as discussed in connection with FIG. 2. The gastric bypass 10 may, in addition to or in place of ribs, preferably include one or more sleeves 250 and wires 252 as discussed in connection with FIG. 3. Many other combinations of the different features discussed herein may be obtained and used in connection with the present invention and the invention should not be limited to those combinations explicitly discussed herein.

The devices of the present invention may preferably be adapted for delivery into the gastrointestinal system endoscopically, although other placement techniques and methods may also be possible. A variety of different delivery methods and structures may be described in, e.g., U.S. Pat. Nos. 4,315,509 (Smit); 4,501,264 (Rockey); and 5,306,300 (Berry); as well as U.S. Patent Publication No. US 2003/0040804 A1 (Stack et al.).

The devices of the present invention may be manufactured of a variety of materials, although the materials used in the devices may preferably be compatible with long-term exposure to ingested food, gastrointestinal fluids (e.g., low pH stomach fluids and high pH intestinal fluids), mechanical stresses associated with the gastrointestinal system, etc. Examples of some suitable materials for the gastric bypass, expandable chambers, esophageal extensions, small bowel extensions, etc., may include, but are not limited to, polymeric materials (e.g., silicone elastomers, polyethylenes, polyether polyurethanes (e.g., TEGADERM), polytetrafluoroethylenes (PTFE), and other materials. If metallic materials such as wires, struts, meshes, etc. are incorporated into the devices of the present invention, they may include, e.g., shape memory metals (such as nickel-titanium alloys), stainless steel, etc. In some instances, the devices of the present invention may include shape memory polymers for one or more different components such as the expandable chambers, etc.

The expandable chambers of the different devices according to the present invention may expand due to mechanical forces associated with wires or other structural members made of stainless steel, shape memory metals, shape memory polymers, etc. In other embodiments, the expandable chambers may be expanded by inflation with a fluid, e.g., saline, air, etc. If a liquid is used for inflation, it may desirably include a dye to provide the patient with an indication that one or more of the chambers has ruptured or is otherwise leaking.

In still other embodiments, the expansion may be provided by gel or polymer-based structural material (e.g., foam, etc.) that, as delivered, is uncured but is expanded/cured when the device is in place in the gastrointestinal system. For example, with the device in location within a patient, the expandable chamber could be expanded and then filled with a solution containing a non-toxic activating agent to expand and fix the structural material. The expanded material within the expandable chamber would preferably provide structural rigidity to resist collapse and preferably also induce satiation. In some embodiments, the expanded material may preferably be biodegradable and/or bioresorbable such that it could slowly erode over time to assist in, e.g., endoscopic removal of the device.

One potentially suitable expandable structural material may be a polylactic acid polymer (PLA, e.g., poly-DL-lactide, etc.) which may be provided as a liquid when dissolved in a solvent such as NMP (N-methyl-2-pyrrolidone), but hardens into a pliable structural material when the NMP diffuses out of the polymer mixture. Both NMP and the polymer PLA are generally considered to be inert and bioresorbable for use within the human (or animal) body. Other expandable structural materials may be known to those skilled in the art.

As used herein and in the appended claims, the singular forms “a,” “and,” and “the” include plural referents unless explicitly limited to the singular form or the context clearly dictates otherwise.

All references and publications cited herein are expressly incorporated herein by reference in their entirety into this disclosure. Illustrative embodiments of this invention are discussed and reference has been made to possible variations within the scope of this invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. An implantable gastrointestinal device comprising:
a gastric bypass comprising an inlet and an outlet;
an expandable chamber attached to the gastric bypass;
an optional esophageal extension attached to the inlet of the gastric bypass, wherein food passes into the gastric bypass through the esophageal extension;
an optional small bowel extension attached to the outlet of the gastric bypass, wherein the small bowel extension receives material exiting the outlet of the gastric bypass.

2. A device according to claim 1, wherein the gastric bypass comprises a flaccid gastric bag.

3. A device according to claim 1, wherein the gastric bypass comprises ribbing molded therein, wherein the ribbing maintains an open volume in the gastric bypass in the absence of compressive forces thereon.

4. A device according to claim 1, wherein the gastric bypass comprises a sleeve containing a wire, wherein the sleeve and wire cooperate to maintain an open volume in the gastric bypass in the absence of compressive forces thereon.

5. An implantable gastrointestinal device comprising:
a gastric bypass comprising an inlet and an outlet;
an expandable chamber attached to an outer surface of the gastric bypass, wherein the expandable chamber comprises a plurality of adjacent subchambers in fluid communication with each other, wherein the plurality of adjacent subchambers are distributed over at least a portion of the outer surface of the gastric bypass, and wherein the plurality of adjacent subchambers maintain an open volume in the gastric bypass in the absence of compressive forces thereon;
an optional esophageal extension attached to the inlet of the gastric bypass, wherein food passes into the gastric bypass through the esophageal extension;
an optional small bowel extension attached to the outlet of the gastric bypass, wherein the small bowel extension receives material exiting the outlet of the gastric bypass.

6. A device according to claim 5, wherein the plurality of adjacent subchambers are arranged in a quasi-geodesic pattern.

7. An implantable gastro-intestinal device comprising:
a gastric bypass comprising an inlet and an outlet;
an expandable toroidal chamber located proximate the inlet of the gastric bypass, wherein the toroidal chamber holds the inlet in an open configuration when the toroidal chamber is inflated;
an optional esophageal extension attached to the inlet of the gastric bypass, wherein food passes into the gastric bypass through the esophageal extension;
an optional small bowel extension attached to the outlet of the gastric bypass, wherein the small bowel extension receives material exiting the outlet of the gastric bypass.

8. A device according to claim 7, wherein the gastric bypass is flaccid outside of the toroidal chamber.

9. A device according to claim 7, wherein the toroidal chamber is asymmetric.
